(19) United States
(12) Patent Application Publication
(10) Pub. No.: US 2018/0190388 A1
(43) Pub. Date: Jul. 5, 2018

(54) Method and Apparatus to Provide a Virtual Workstation With Enhanced Navigational Efficiency

(71) Applicants: University of Maryland, Baltimore, Baltimore, MD (US); University of Maryland Faculty Physicians, Inc., Baltimore, MD (US)

(72) Inventors: Reuben Mezrich, Baltimore, MD (US); Wayne LaBelle, Baltimore, MD (US)

(21) Appl. No.: 15/736,939

(22) PCT Filed: Jun. 15, 2016
(86) PCT No.: PCT/US16/37600

§ 371 Date: Dec. 15, 2017

Related U.S. Application Data
(60) Provisional application No. 62/175,490, filed on Jun. 15, 2015.

(51) Int. Cl. G16H 80/00; H04N 7/15; G09G 5/14; G06F 3/14; G06F 3/01

(52) U.S. Cl. CPC G16H 80/00; H04N 7/15; G09G 5/14; G06F 3/0487; G06F 3/012; G09G 2354/00; G06F 3/1462

(57) ABSTRACT

A method and apparatus are provided for enhancing a navigational efficiency of a virtual workstation. The method includes receiving a design space describing a desired arrangement of virtual monitors. The method further includes receiving data associated with head movement of a user wearing a virtual reality headset. The method further includes determining a movement of a view space over the design space where the view space encompasses only a portion of the design space. The view space movement is based on the head movement such that a ratio of the view space movement to the head movement is in a range comprising values other than unity. The method also includes moving the view space based on the determined movement of the view space and presenting on the virtual reality headset the portion of the design space within the view space.

### CROSS-REFERENCE TO RELATED APPLICATIONS

This application claims benefit of Provisional Appln. 62/175,490, filed Jun. 15, 2016, the entire contents of which are hereby incorporated by reference as if fully set forth herein, under 35 U.S.C. § 119(e).

### BACKGROUND

A radiology reading room is an environment where radiologists view images and data on multiple monitors. It is convenient for the reading room to include a large number of monitors in various arrangements, with dedicated monitors to display different content such as images, data and descriptive text that are used during the radiology diagnostic process.

### SUMMARY

It is here recognized that conventional radiology reading rooms with a large amount of monitors are deficient, since they require a large amount of financial resources to acquire the monitors and a large amount of physical space to subsequently position the monitors in the reading room. Additionally, once a specific arrangement of the monitors in the reading room is set, even small adjustments to the arrangement may involve extensive steps, including repositioning a substantial number of the monitors. Additionally, when a user moves their head from a first monitor to a second monitor in the arrangement, the user is required to move their head by the same angle that separates the first and second monitors. This requirement reduces the work efficiency of a user performing radiology diagnosis.

In a first set of embodiments, a method is provided for enhancing a navigational efficiency of a virtual workstation. The method includes receiving, on a processor, a design space describing a desired arrangement of virtual monitors. The method further includes receiving, on the processor, data associated with head movement of a user wearing a virtual reality headset. The method further includes determining, on the processor, a movement of a view space over the design space where the view space encompasses only a portion of the design space and where the movement of the view space is based on the head movement such that a ratio of the view space movement to the head movement is in a range comprising values other than unity. The method also includes moving the view space over the design space based on the determined movement of the view space and presenting on the virtual reality headset the portion of the design space within the view space.

In a second set of embodiments, an apparatus is provided for enhancing a navigational efficiency of a virtual workstation. The apparatus includes a virtual reality headset configured to be worn on a user's head. The apparatus also includes a processor and a memory including a sequence of instructions. The memory and the sequence of instructions is configured to, with the processor, cause the apparatus to receive a design space describing a desired arrangement of virtual monitors. The memory and the sequence of instructions is also configured to, with the processor, cause the apparatus to receive data associated with head movement of a user wearing the virtual reality headset. The memory and the sequence of instructions is also configured to, with the processor, cause the apparatus to determine a movement of a view space over the design space where the view space encompasses only a portion of the design space. The movement of the view space is based on the head movement such that a ratio of the view space movement to the head movement is in a range comprising values other than unity. The memory and the sequence of instructions is also configured to, with the processor, cause the apparatus to move the view space over the design space based on the determined movement of the view space and to present on the virtual reality headset the portion of the design space within the view space.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a photograph that illustrates an example of a conventional radiology reading room with a plurality of display devices;

FIG. 1B is a photograph that illustrates an example of a conventional radiology reading room with a plurality of monitors;

FIG. 1C is a photograph that illustrates an example of a conventional radiology workstation with a plurality of monitors;

FIG. 1D is a photograph that illustrates the plurality of monitors of the conventional radiology workstation of FIG. 1C;

FIG. 1E is a block diagram that illustrates an example of a top view of the plurality of monitors of the conventional radiology workstation of FIG. 1D;

FIG. 2A is a block diagram that illustrates an example of a system for enhancing a navigational efficiency of a virtual workstation, according to an embodiment;

FIG. 2B is a photograph that illustrates an example of a virtual reality headset of the system of FIG. 2A, according to an embodiment;

FIG. 2C is a photograph that illustrates an example of the virtual reality headset of FIG. 2B removed from the user, according to an embodiment;

FIG. 2D is a block diagram that illustrates an example of a system for enhancing a navigational efficiency of a virtual workstation, according to an embodiment;

FIG. 3 is a flow diagram that illustrates an example of a method for enhancing a navigational efficiency of a virtual workstation, according to an embodiment;

FIG. 4A is a block diagram that illustrates an example of a design space, according to an embodiment;

FIG. 4B is a block diagram that illustrates an example of a view space over a first virtual monitor of the design space in FIG. 4A, according to an embodiment;

FIG. 4C is a block diagram that illustrates an example of the view space over a second virtual monitor of the design space in FIG. 4A, according to an embodiment;

FIG. 4D is a block diagram that illustrates an example of a side view of the design space of FIG. 4A with respect to the user, according to an embodiment;

FIG. 4E is a block diagram that illustrates an example of a top view of the design space of FIG. 4A with respect to the user, according to an embodiment;

FIG. 4F-FIG. 4G are block diagrams that illustrate an example of respective first and second design spaces of first and second virtual reality headsets connected over a network, according to an embodiment;

FIG. 4H-FIG. 4I are block diagrams that illustrate an example of respective first and second matching design spaces of first and second virtual reality headsets connected over a network, according to an embodiment;

FIG. 5A is a block diagram that illustrates an example of data flow within the system of FIG. 2D, according to an embodiment;

FIG. 5B is a block diagram that illustrates an example of data flow among components within the system of FIG. 2D, according to an embodiment;

FIG. 6 is a block diagram that illustrates a computer system upon which an embodiment of the invention may be implemented; and

FIG. 7 is a block diagram that illustrates a chip set upon which an embodiment of the invention may be implemented.

### DETAILED DESCRIPTION

A method and apparatus are described for enhancing a navigational efficiency of a virtual workstation. For purposes of the following description, a workstation is defined as one or more monitors arranged in a particular spatial arrangement, where each monitor has a particular size and a particular position within the spatial arrangement and displays selective content that is viewed side by side by a user of the workstation. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of virtual workstations and enhancing a navigational efficiency of a virtual workstation, including a virtual radiology workstation. However, the invention is not limited to this context. In other embodiments users review the totality of patient information (lab values, medical charts, videos of intra-operative procedures which require multiple monitors). In other embodiments, other workstations made up of multiple monitors are used, such as exchanges where activity on multiple markets and multiple stocks are viewed at once; air traffic controller rooms; power utility control rooms where electric usage and generation over large areas area monitored; security centers where monitors display activity from multiple sites; military installations such as North American Aerospace Defense Command (NORAD) where the theaters of various forces are monitored; among others.

### 1. Overview

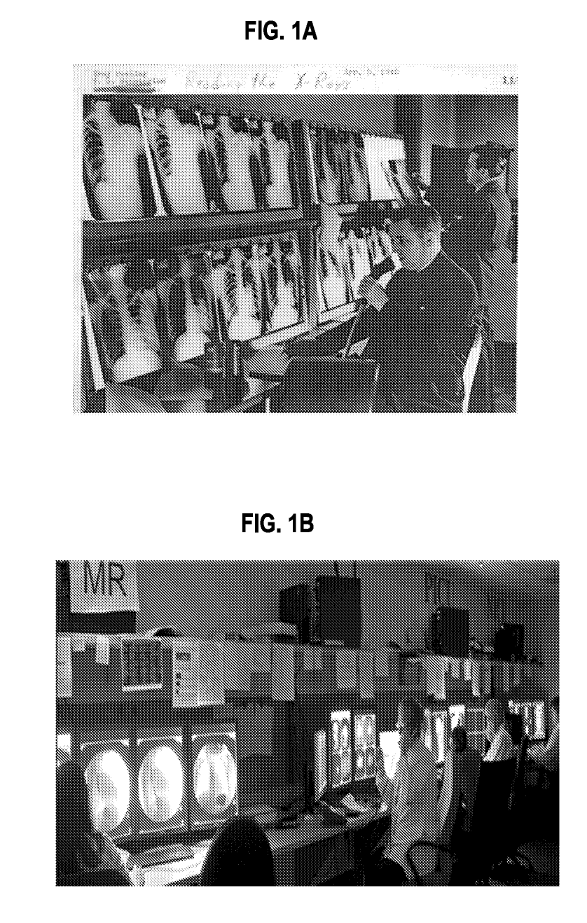

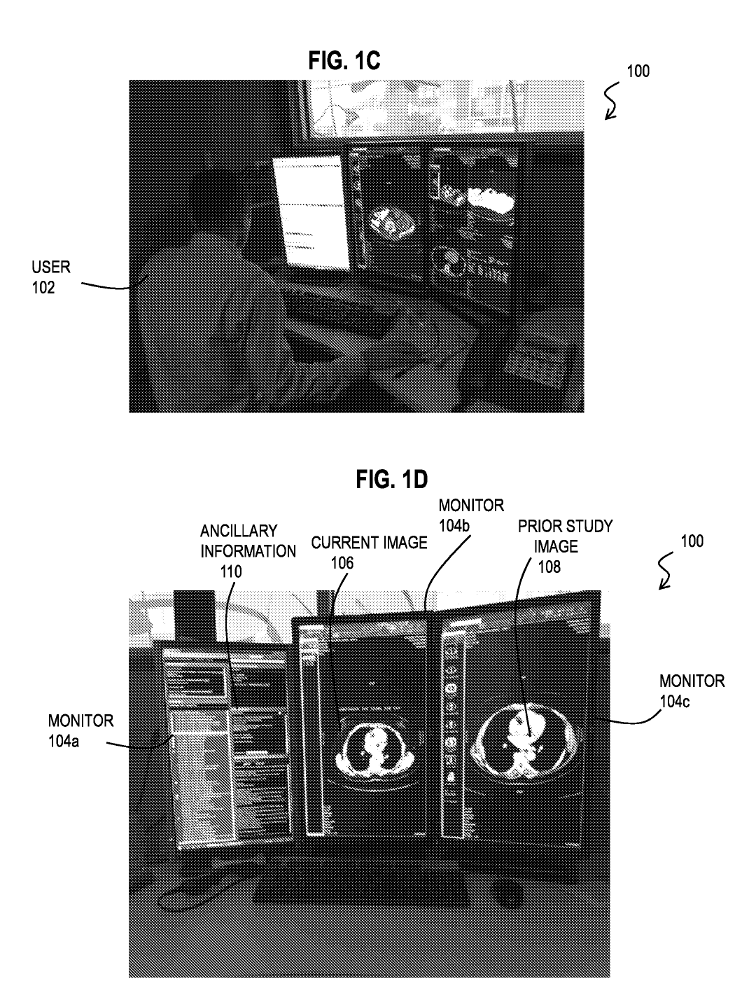

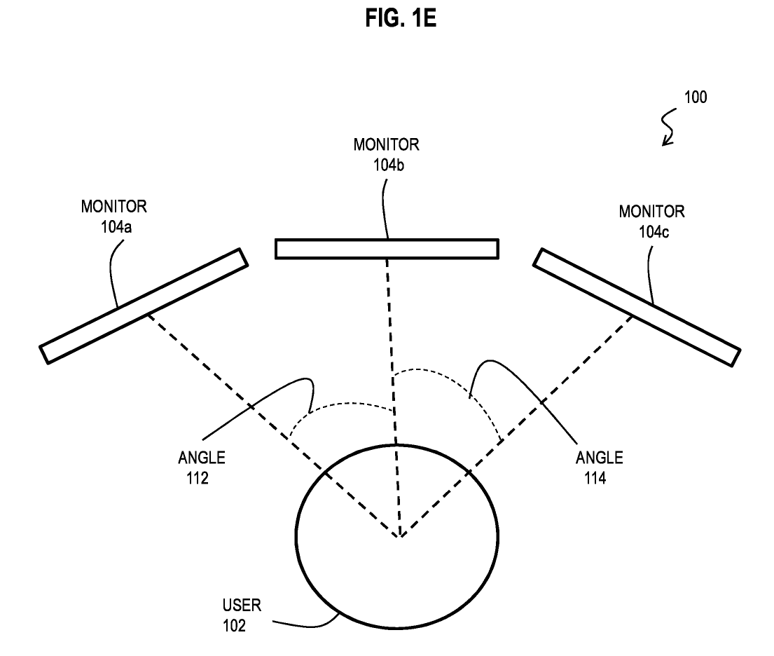

FIG. 1A is a photograph that illustrates an example of a conventional radiology reading room with a plurality of display devices including a plurality of individual films positioned on an illuminator (e.g. light wall or light table). The individual films serve as storage media for image data and the illuminator displays each image by transmitting light from the illuminator through the film. The conventional reading room includes several films spread out over an illuminator so that a user of the reading room can view multiple radiology images as the user shifts his or her head position from one film to the next.

FIG. 1B is a photograph that illustrates an example of a conventional radiology reading room with a plurality of monitors that display radiology images and large data banks that store image data. The large quantity of monitors and data banks involve considerable financial cost to acquire. Additionally, the monitors and data banks line the reading room and thus involve considerable physical space to house. As with the conventional reading room of FIG. 1A, a user of the reading room can view multiple radiology images and associated text, as the user shifts his or her head position to direct his or her gaze from one monitor or set of monitors to the next.

FIG. 1C-FIG. 1D are photographs that illustrate an example of a conventional radiology workstation 100 for a user 102 with a plurality of monitors 104a-104c. FIG. 1D illustrates the workstation 100 from the perspective of the user 102. In the workstation 100, a current image 106 is displayed on a center monitor 104b and is the image 106 that is to be reviewed, diagnosed and reported by the user 102. Additionally, a prior study image 108 is displayed on a right monitor 104c and corresponds to a prior patient study and is used to compare with the current image 106. Additionally, non-image ancillary information 110 (e.g. text and graphs indicating prior studies, clinical notes, etc.) is displayed on a left monitor 104a.

The conventional reading rooms of FIG. 1A-FIG. 1B and conventional workstation 100 of FIG. 1C-FIG. 1D have notable drawbacks. For example, they each involve a fixed arrangement of display devices (e.g. monitors) where each display device has a fixed size and fixed position and thus cannot be easily reconfigured without involving substantial steps. In another example, the conventional reading rooms and conventional workstation are not portable and thus the radiologist is confined to working in the physical location of the reading room or workstation. In another example, the conventional reading rooms and conventional workstation involve substantial financial cost to acquire the display devices and further involve substantial physical space to house the display devices. Consequently, it would be advantageous to provide a workstation that addressed one or more of these drawbacks of conventional workstations. For example, it would be advantageous to provide a workstation that permits the radiologist to work in other environments, such as home, boat, hotel room, vacation house etc. Thus, there is a need for methods and systems enabling radiologists to work from various places in the world (e.g. home, boat, hotel room, vacation house etc.) without the need of multiple large computer displays and dedicated office space.

FIG. 1E is a block diagram that illustrates an example of a top view of the plurality of monitors 104a, 104b, 104c of the conventional radiology workstation 100 of FIG. 1D. The left monitor 104a and center monitor 104b have an angular separation of an angle 112 and the center monitor 104b and right monitor 104c have an angular separation of an angle 114. If the user 102 is observing the ancillary information 110 on the left monitor 104a and wants change the view and observe the current image 106 on the center monitor 104b, the user 102 must rotate his or her head clockwise by the angle 112. Similarly, if the user 102 is observing the current image 106 on the center monitor 104b and wants to change the view and observe the prior study image 108 on the right monitor 104c, the user 102 must rotate his or her head clockwise by the angle 114. Thus, a movement of the head (e.g. angular rotation) must equal a movement of the view (e.g. angular separation between monitors). A drawback of this arrangement is that over time, the user 102 is required to rotate his or her head by large angular amounts, which leads to work inefficiencies. For example, every time the user 102 wants to change the view from the left monitor 104a to the right monitor 104c, the user 102 must rotate his or her head by a sum of the angle 112 and the angle 114.

Thus, it would be advantageous to provide a workstation where the user 102 was not required to rotate his or her head as much as in the conventional workstation 100 while achieving the same change in view. For example, it would be more efficient if the user 102 could change the view from the left monitor 104a to the center monitor 104b by rotating his or her head by an angle that is less than the angle 112. In another example, it would be more efficient if the user 102 could change the view from the center monitor 104b to the right monitor 104b by rotating his or her head by an angle that is less than the angle 114.

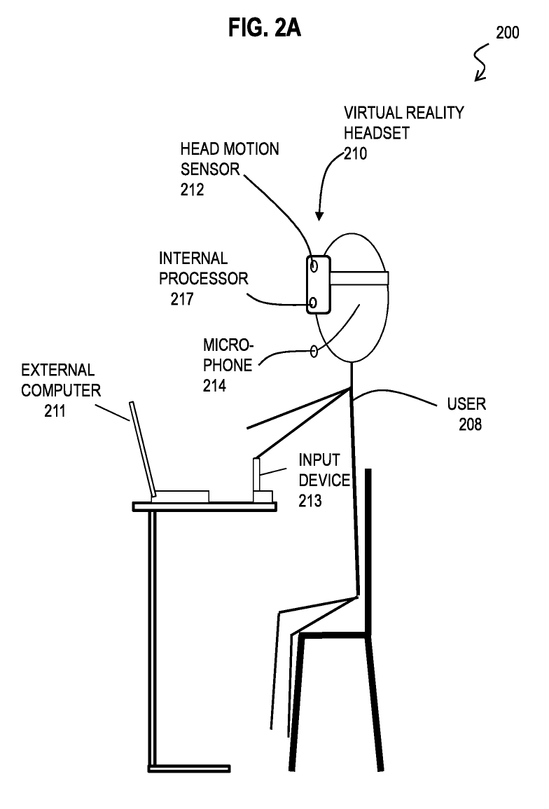

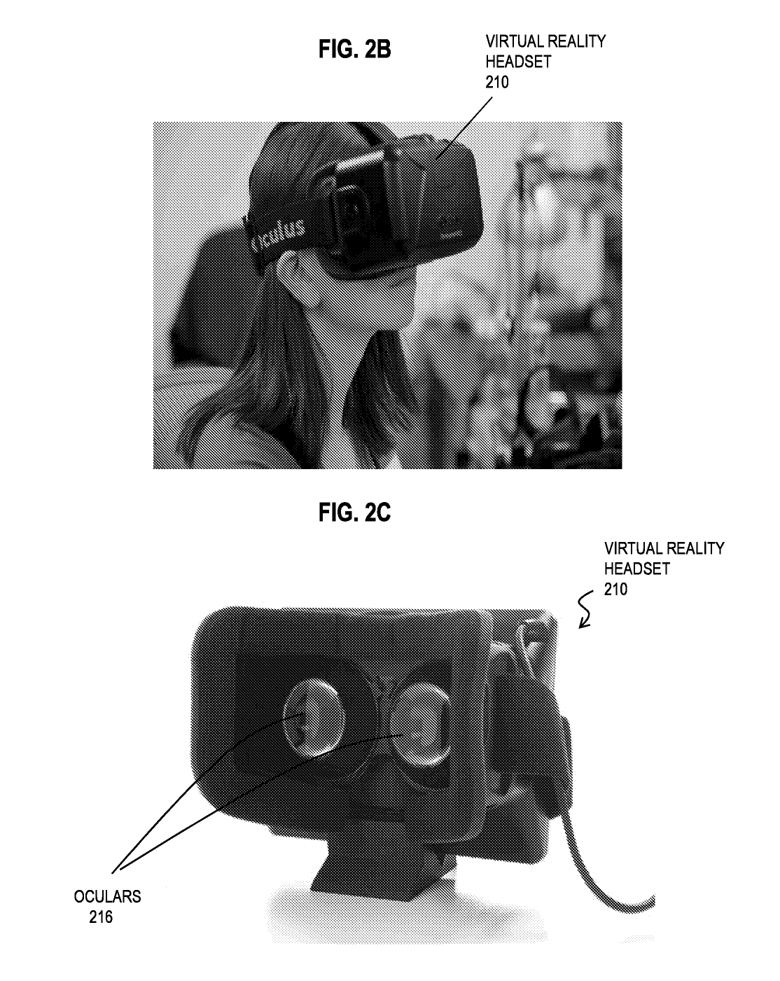

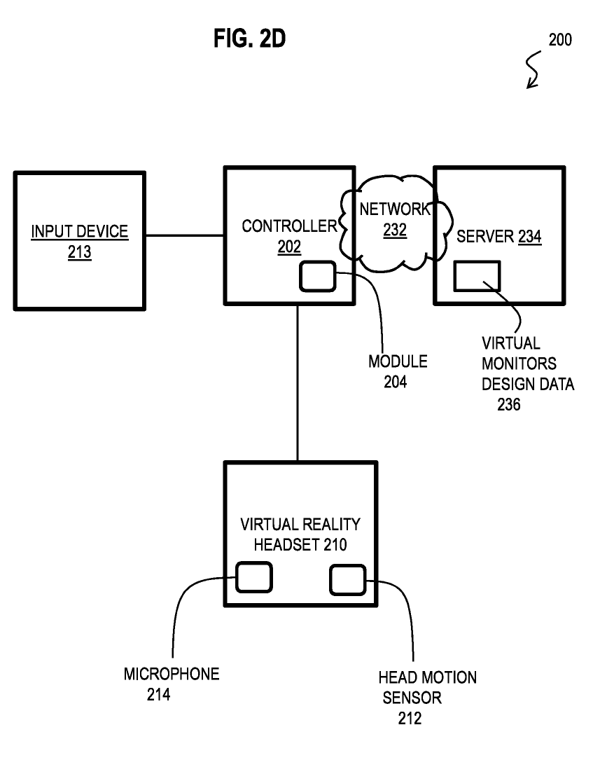

FIG. 2A is a block diagram that illustrates an example of a system 200 for enhancing a navigational efficiency of a virtual workstation, according to an embodiment. The system 200 includes a virtual reality headset 210 worn on a head of the user 208. Any virtual reality headset with sufficient pixel resolution can be used. In various embodiments, the virtual reality headset 210 is Oculus Rift® developed by Oculus VR, LLC, of Menlo Park, Calif.; Oculus Rift, HTC Vive, Razer OSVR, Sony Playstation VR, Samsung Gear VR, Microsoft Hololens, Homido, Google Cardboard, Zeiss VR One, FOVE VR, among others. In some embodiments, the virtual reality headset is merely a mount for a smart phone that serves as the view screen and headset processor. The user 208 is not part of the system 200. FIG. 2B-FIG. 2C are photographs that illustrate an example of the virtual reality headset 210 worn by the user 208, according to an embodiment. The virtual reality headset 210 is any high resolution headset currently available on the market. The virtual reality headset 210 includes two oculars 216 where each ocular 216 is configured to display separate for each eye a 2D image to stereoscopically recreate a 3D image of a radiology workstation or an image presented on one or more monitors therein.

The current level of technology in virtual reality, computer gaming, and sensing is sufficiently sophisticated and mature such that a person of ordinary skill in the above arts would know how to technically implement the inventions described in this application. The resolution capabilities of the available virtual reality headsets is more than adequate for diagnostic quality display. For example Computed Tomography (CT) images have a resolution of less than 1000×1000 pixels. Magnetic Resonance Imaging (MRI) images have resolutions of less than 500×500 pixels, Ultrasound images have resolutions of less than 256×256 pixels and Nuclear Medicine images are typically less than 125×125 pixels. The headset resolution is typically 1000×2000, which is more than adequate. The one caveat would be mammography, in which the Food and Drug Administration (FDA) has mandated that the images be viewed on 5 megapixel monitors for diagnosis, which is more than the capability of the current headsets. In such embodiments, the virtual headset is used to display only a part of the mammogram that can fit in the pixels available, when viewed at full resolution. In other embodiments, diagnosis of the mammogram is performed on another display device (e.g. that conforms with the FDA mandate) after which a clinician (or the patient) can view the same mammogram on the virtual reality headset if desired. In most cases, the resolution of the mammogram on the virtual reality headset will be sufficient to appreciate the disease. In some embodiments, the image is viewed selectively at either full or at degraded resolution based on operation of the system 200. There is no such high resolution mandate for other diagnostic images (e.g. there is no such mandate for “plain films”).

The current headsets are not heavy and are designed to be worn for hours at a time. The virtual radiology system as a whole is highly portable since all its components (e.g. headset, computer, microphone, recorder, position sensors, cameras etc.) are not heavy and are small. All the above components can be miniaturized. For example, the components may be stored/presented as a kit in a dedicated small and light briefcase. One of the most important features of the virtual radiology workstation is that it does not require a dedicated workplace (e.g. a reading room) or a dedicated environment. They can be used anywhere and provide their own environment. The virtual radiology system is relatively inexpensive (costs for the headset now range from about $20 to about $1000) with the lower cost units employing the user's smartphone and the higher cost units providing a built in display. All have about the same resolution. The cost is low especially as compared to conventional radiology workstations which are 10 to 20 times more expensive.

In some embodiments, the virtual reality headset 210 includes a motion sensor 212 configured to measure one or more parameters relating to a position or movement of the head of the user 208. In other embodiments, the motion sensor 212 is separate from the virtual reality headset 210. In an example embodiment, the motion sensor 212 determines, in real-time, the position, angulation and/or motion of the user's 208 head and transmits, in real-time, data corresponding to such position and motion to a processor, such as a processor on an external computer 211 or an internal processor 217 within the virtual reality headset 210, or some combination. In some embodiments, the external computer 211 is one of a laptop, a tablet, a smart-phone, a miniature computer, or any other suitable computer. In some embodiments, a screen or monitor on the separate computer 211 is not needed or is disabled. In some embodiments, the external computer 211 or internal processor 217 are configured to receive the parameters relating to the position of movement of the head of the user 208 from the motion sensor 212 and are further configured to determine the position or movement of the head based on these parameters. In the less expensive versions of the virtual reality headset, motion sensing is accomplished by the electronics built into most smartphones. In the expensive versions of the virtual reality headset, a separate motion sensor is used. The results are substantially the same.

In some embodiments, the external computer 211 or internal processor 217 are configured to provide images or data to the virtual reality headset 210, to cause the virtual reality headset 210 to display the images and data, based on the determined position or movement of the user's 202 head. The displayed images and data on the virtual reality headset 210 enable the user 202 (e.g. radiologist) to perform a job function (e.g. diagnosis).

In some embodiments, the system 200 includes a microphone 214 connected to a recording device (not shown) to enable the user 208 to record his/her observations and notes regarding the images displayed on the virtual reality headset 210 at the time the user 208 analyzes the images. However, the system 200 need not include the microphone 214.

In some embodiments, the system 200 includes an input device 213 configured to enable the user 208 to change displayed content on the virtual reality headset 210 according to the user's 208 needs. In an example embodiment, the user 208 uses the input device 213 to control and act on content displayed on virtual monitors within a view space of the virtual reality headset 210. In an example embodiment, the input device 213 is used to scroll through sets of image slices of a computed tomography (CT) scan, a magnetic resonance imaging (MRI) scan, a positron emission tomography (PET) scan or an ultrasound scan. In another example embodiment, the input device 213 is used to change a scale or view angle of an image. In another example embodiment, the input device 213 is used to browse through text displayed on the virtual monitor within the view space of the virtual reality headset 210. In another example embodiment, the input device 213 is used to select text and parts of an image on the virtual monitor within the view space of the virtual reality headset 210. In another example embodiment, the input device 213 is used to browse images and data corresponding to different patients. In an example embodiment, the input device 213 is a keyboard, a mouse, or a joystick or any similar device that is configured for the user 208 to provide input to the computer. In some embodiments, the input device is wireless, (e.g. using Bluetooth technology). In some embodiments, the input device is used to arrange one or more virtual monitors in a design space to be viewed one viewable portion at a time as the user 208 moves his or her head.

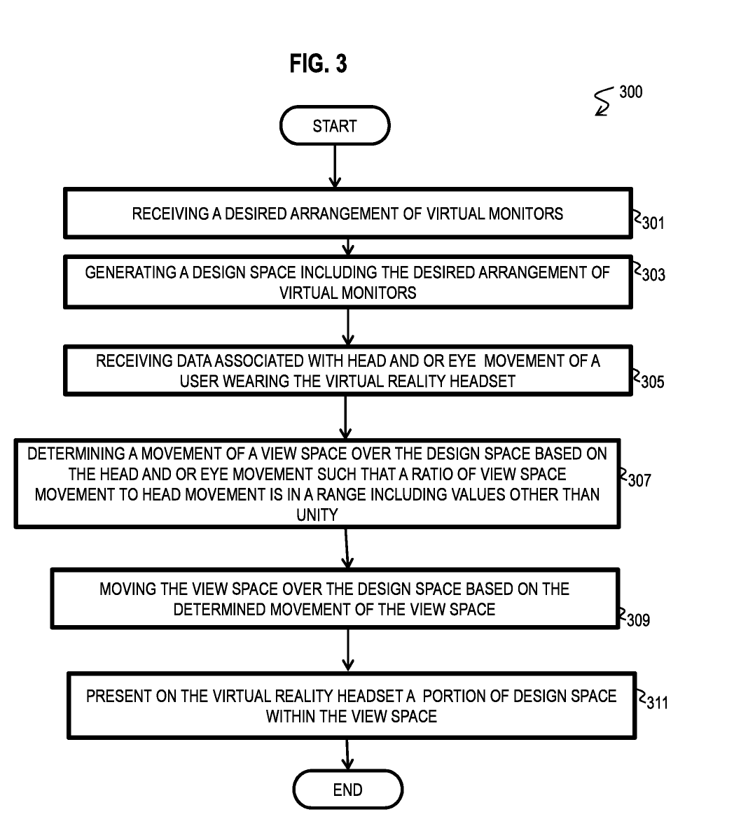

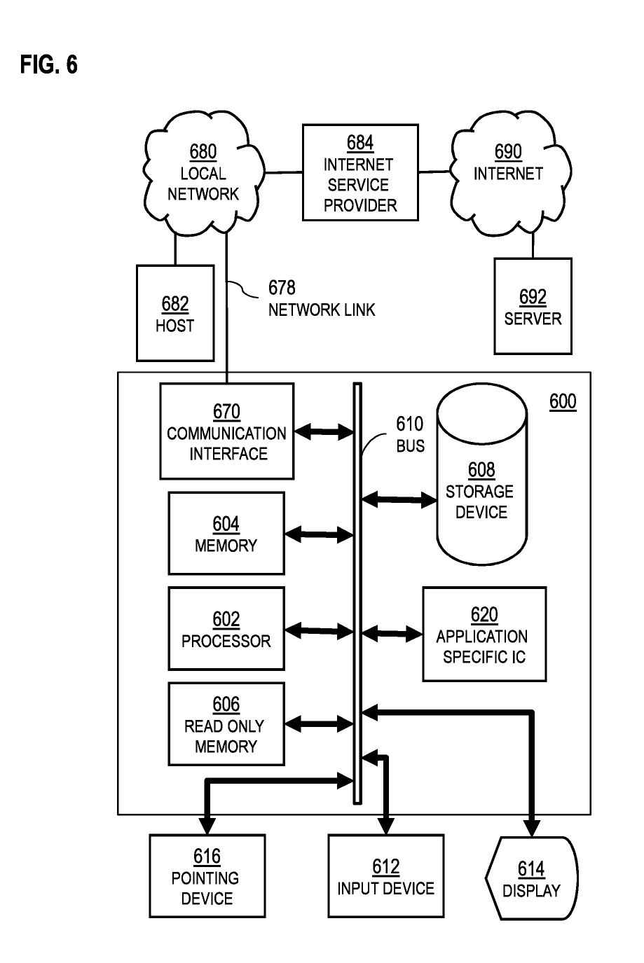

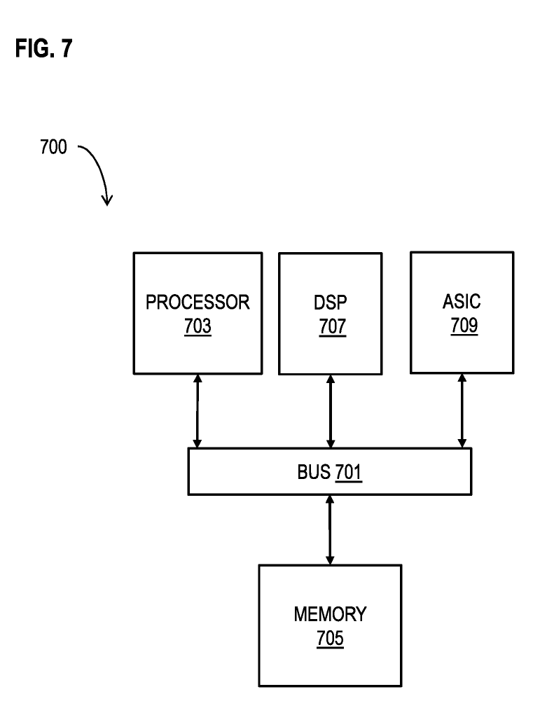

FIG. 2D is a block diagram that illustrates an example of a system 200 for enhancing a virtual workstation, according to an embodiment. In the illustrated embodiment, the system includes the input device 213, the virtual reality headset 210, the microphone 214 and the head motion sensor 212, described above. The system 200 includes a controller 202 with a module 204 that causes the controller 202 to perform one or more steps as discussed below. In various embodiments, the controller 202 comprises a general purpose computer system, as depicted in FIG. 6 or a chip set as depicted in FIG. 7, and instructions to cause the computer or chip set to perform one or more steps of a method described below with reference to FIG. 3. In some embodiments, the controller 202 is positioned within the external computer 211. In other embodiments, the controller 202 is positioned within the internal processor 217 or some combination. In the illustrated embodiment, the controller 202 communicates with a remote server 234 via a communications network 232. In some embodiments, one or more steps of the method described in FIG. 3 are performed by the server 234. The system includes data storage for data 236 that indicates a design of the virtual monitors in a design space, as described in more detail below. In the illustrated embodiment, the design data 236 is stored on the remote server 234, but in other embodiments, the data is stored on or with the controller 202 on the external local computer 211 or internal processor 217 or some combination.

FIG. 3 is a flow diagram that illustrates an example of a method 300 for enhancing a navigational efficiency of a virtual workstation, according to an embodiment. Although steps are depicted in FIG. 3 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 301, a desired arrangement of virtual monitors, e.g. in virtual monitors design data 236, is received by the controller 202. In some embodiments, during step 301, the user 208 inputs one or more parameters of the desired arrangement of virtual monitors using the input device 213. In an example embodiment, the parameters include one or more of a number of virtual monitors in the desired arrangement; a size of each virtual monitor in the desired arrangement; a position of each virtual monitor in the desired arrangement and a desired content type to be displayed on each virtual monitor. Additionally, in other embodiments, during step 301, the user 208 inputs one or more parameters of a modification to the desired arrangement of virtual monitors using the input device 213. In an example embodiment, the parameters include one or more of a modification to the number of virtual monitors in the desired arrangement; a modification to the size of one or more virtual monitors in the desired arrangement; a modification to the position of one or more virtual monitors in the desired arrangement and a modification to the desired content type to be displayed on one or more virtual monitors. In other embodiments, the desired arrangement of virtual monitors is received by the controller 202 from an external source other than the user 208. In an example embodiment, the desired arrangement of virtual monitors is received through a network 232 from a server 234, such as a second controller of a second system that is similar to the system 200, where the controller 202 and the server 234 are connected over the network 232. The number of virtual monitors and their size(s) and their position(s) in virtual space, or the contents or set of contents for each, or some combination, can be determined by the user and kept as a user preference, e.g. on the server 234.

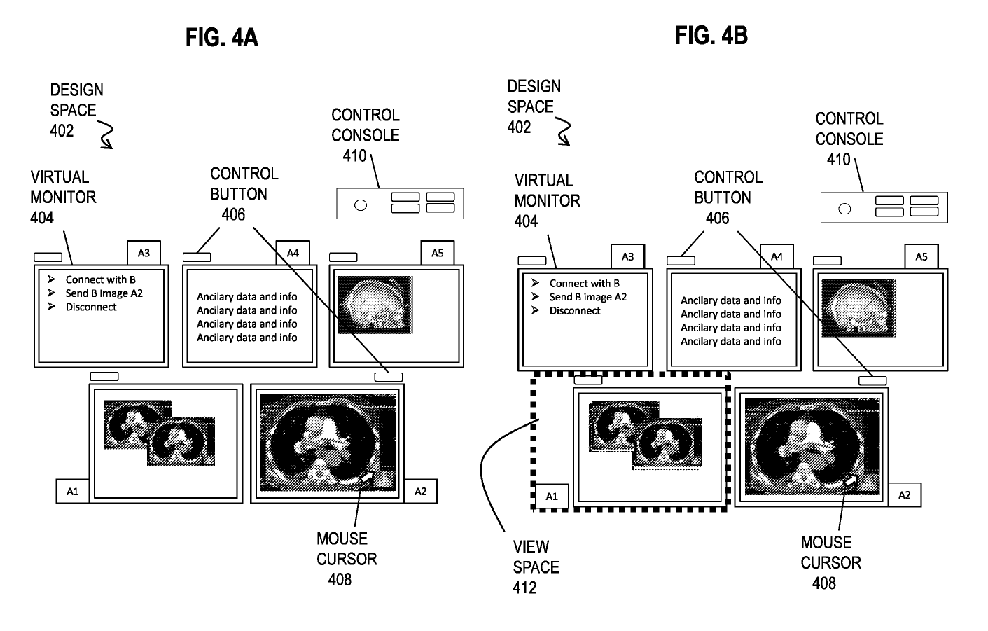

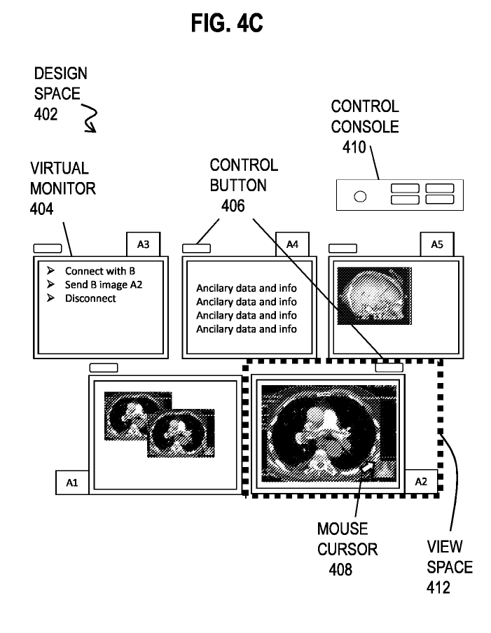

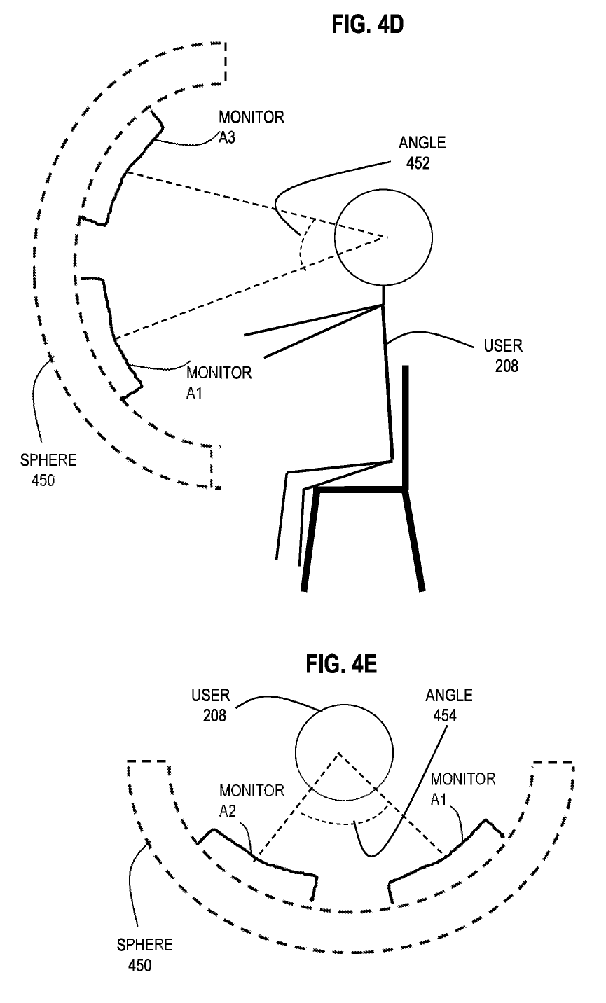

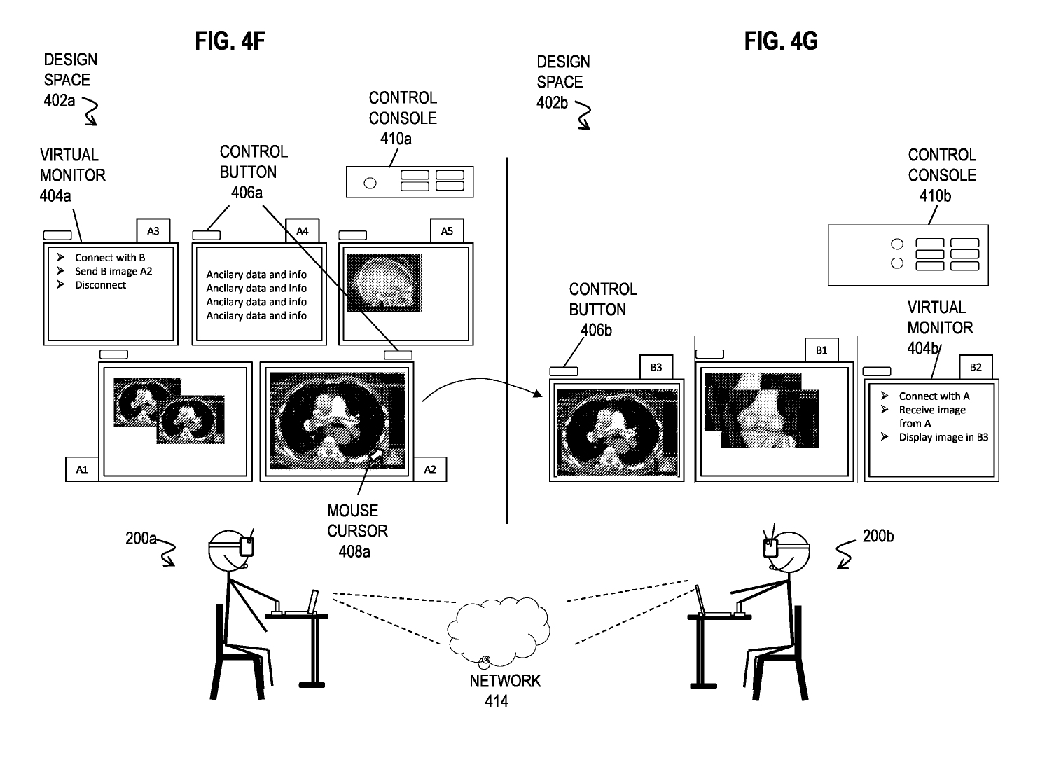

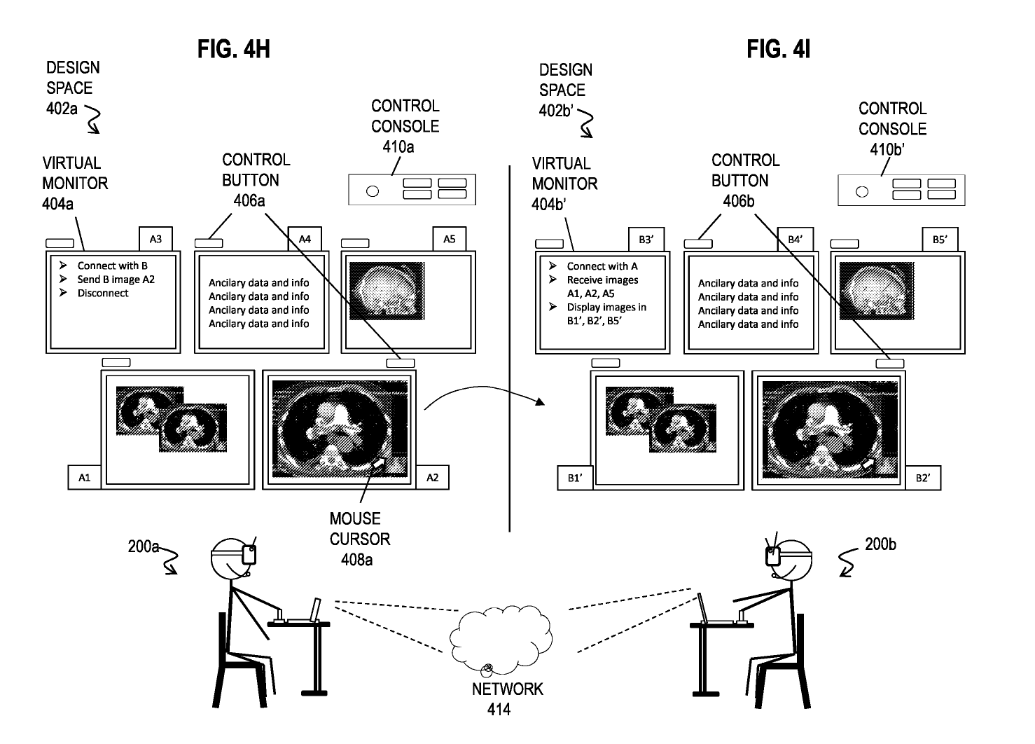

In step 303, data 236 indicating a design space 402 is generated based on the desired arrangement of virtual monitors input at step 301. In some embodiments, the design space 402 is stored in a memory of the controller 202 or on the remote server 234 as depicted in FIG. 2D. FIG. 4A is a block diagram that illustrates an example of a design space 402, according to an embodiment. In an example embodiment, the depicted design space 402 is based on an inputted desired arrangement at step 301 including a desired number (e.g. five) of virtual monitors 404 (A1-A5), a desired size (e.g. approximately equal) of each virtual monitor 404, a desired positional arrangement (e.g. two horizontal rows) of the virtual monitors 404 and a desired content type (e.g. three monitors to display image content, two monitors to display text) for each virtual monitor 404.

Additionally, in some embodiments, the design space 402 includes a control button 406 for each virtual monitor 404 that permits the user 208 to support action relating to the specific virtual monitor 404. In an example embodiment, the control button 406 is used to select a specific virtual monitor 404 (e.g. the virtual monitor 404 within the view space that the user 208 is observing) such that the input device 213 only affects content on that specific virtual monitor 404. A cursor 408 is depicted for the input device 213. Additionally, in some embodiments, a control console 410 is provided, that includes various color codes associated with different functions of the control button 406. In an example embodiment, if the user 208 wants to select virtual monitor A2, the user 208 moves the cursor 408 over the color code on the control console 410 (e.g. red) associated with selecting a specific virtual monitor 404, clicks this color code and subsequently clicks the control button 406 for the virtual monitor A2. In some embodiments, the system 200 gives focus to whichever monitor is being viewed, as described below by the view space 412. The view space 412 is the portion of the design space 402 that can be displayed on the virtual reality headset (e.g. the 1000×2000 pixels displayed on most current virtual reality headsets). In these embodiments, the system 200 selects the specific virtual monitor based on identifying the virtual monitor within the view space 412 (e.g. the virtual monitor being viewed by the user) such that any user operation (e.g. scrolling, zooming, annotation, etc.) only affects content on that specific virtual monitor.

During step 303, the controller 202 receives the inputted parameters of the desired arrangement inputted during step 301. The module 204 then processes the inputted parameters and generates the design space 402 based on the inputted parameters. In some embodiments, the design space 402 is stored in a memory of the controller 202 or remote server as design data 236.

In step 305, data associated with head movement of the user 208 wearing the virtual reality headset 210 is received by the controller 202. In some embodiments, during step 305, the motion sensor 212 determines, in real-time, the position, angulation and/or motion of the user's 208 head and transmits, in real-time, data corresponding to such position and motion to the controller 202. The module 204 then processes the position, angulation and/or motion data from the motion sensor 212 to determine head movement of the user 208.

In step 307, movement of a view space 412 over the design space 402 is determined, based on the head movement determined in step 305. FIG. 4B-FIG. 4C are block diagrams that illustrate an example of movement of the view space 412 from a first virtual monitor A1 (FIG. 4A) of the design space 402 to a second virtual monitor A2 (FIG. 4B), according to an embodiment. The view space 412 is a portion of the design space 402 that can be displayed at one time on the virtual reality headset. The view space 412 moves over the design space 402 based on the head movement of the user 208 determined in step 305. The view space 412 represents a portion of the design space 402 that is visible to the user, based on the head position of the user. In an example embodiment, the view space 412 is a rectangular area, however the view space 412 is not limited to any particular shape. In some embodiment, the view space is set to display more of the design space in the view space at lower resolution or to display a smaller portion of the design space at full resolution. Thus in various embodiments, the percentage of the design space within the view space is selectable, e.g., the view space can appear to be larger or smaller than depicted in FIG. 4B through FIG. 4C.

In some embodiments, during step 307, the module 204 determines the movement of the view space 412 over the design space 402 based on the head movement of the user 208 determined in step 305. In an example embodiment, the module 204 determines the movement of the view space 412 such that a ratio of the view space movement to the head movement determined in step 305 is in a range including values other than unity. In an example embodiment, the range values include 50%-150%. In various embodiments, the range is set to whatever the user prefers and is comfortable with. In some embodiments, the ratio is preset or programmable into the module 204. In other embodiments, the ratio is input by the user 208 with the input device 213 and received by the module 204. In the embodiment of FIG. 4A through FIG. 4B, the determined movement of the view space 412 (e.g. from the monitor A1 in FIG. 4A to the monitor A2 in FIG. 4B) is less than or greater than the head movement determined in step 305. In this example embodiment, if the determined head movement in step 305 is X degrees, the determined movement of the view space 412 is Y degrees, where Y<X or Y>X. In an example embodiment, Y>X such that the user 208 advantageously need not move their head entirely from A1 to A2 in order for the view space 412 to move from A1 to A2. In some embodiments, when one looks at a particular window (e.g. “focus” is given) that window can zoom up—enlarge—to enable high detail viewing. This would advantageously reduce head movement even further to allow even more monitors to be placed into the design space. In other embodiments, some headsets (e.g. FOVE) now include eye tracking which would reduce head movement even further.

FIG. 4D is a block diagram that illustrates an example of a side view of the design space 402 of FIG. 4A with respect to the user, according to an embodiment. FIG. 4E is a block diagram that illustrates an example of a top view of the design space 402 of FIG. 4A with respect to the user, according to an embodiment. In some embodiments, the virtual monitors 404 of the design space 402 are arrayed on a virtual sphere 450 (or hemi-sphere) that surrounds the user 208. In other embodiments, the virtual monitors 404 are arrayed on a virtual curved surface having a curvature different than a spherical surface. The monitors A1, A3 are angularly spaced apart by an angle 452 in a vertical plane (FIG. 4D) whereas the monitors A1, A2 are angular spaced apart by an angle 454 in a horizontal plane (FIG. 4E). In this example embodiment, if the user 208 wants to change the view from monitor A1 to A3, in order for the view space 412 to move in a vertical plane from monitor A1 to A3, the user 208 advantageously need only rotate his or her head by a vertical angle that is less than the angle 452. Similarly, in this example embodiment, if the user 208 wants to change the view from monitor A1 to A2, in order for the view space 412 to move in a horizontal plane from monitor A1 to A2, the user 208 advantageously need only rotate his or her head by a horizontal angle that is less than the angle 454.

In step 309, the view space 412 is moved over the design space 402 based on the determined movement of the view space 412 in step 307. During step 307, the module 204 determines the portion of the design space 402 corresponding to the moved view space 412 and stores this portion of the design space 402 in the memory of the controller 202.

In step 311, the portion of the design space 402 corresponding to the moved view space 412 is presented on the virtual reality headset 210. In some embodiments, during step 311, the module 204 retrieves the stored portion of the design space 402 corresponding to the moved view space from step 309 and causes the controller 202 to transmit a signal to the virtual reality headset 210 to render the stored portion of the design space 402.

FIG. 4F through FIG. 4G are block diagrams that illustrate an example of respective first and second design spaces 402a, 402b within first and second virtual reality headsets connected over a network 414, according to an embodiment. Each design space 402a, 402b includes similar features as the design space 402 discussed above. The second design space 402b has a different arrangement of virtual monitors 404b than the desired arrangement of virtual monitors 404a of the first design space 402a. The first user of the first design space 402a can share the content on one or more virtual displays 404a with the second user. In some embodiments, the first user shares content on one or more virtual displays 404a by using the input device 213 to select the control button 406a associated with the one or more virtual monitors 404a. In some embodiments, the user just selects the control button 406a. In some embodiments, the user clicks on the control console 410a. In some embodiments, whoever moves the cursor has control. In an example embodiment, content on the remaining virtual monitors 404a whose control button 406a are not selected remains private and thus the second user cannot view the content on the remaining virtual monitors 404a. In this embodiment, the second user selects one or more virtual monitors 404b to display the content from the shared virtual monitors 404a. In an example embodiment, the first user selects monitor A2 such that the content on monitor A2 is shared with the second user, and content on the remaining monitors A1, A3, A4 and A5 is kept private from the second user. In this example embodiment, the second user selects virtual monitor B3 to display the content displayed on virtual monitor A2. In some embodiments, a virtual monitor A3, B2 in each design space 402a, 402b lists action items associated with each design space 402a, 402b. In an example embodiment, the virtual monitor A3 lists action items associated with the first virtual reality headset, including the connection with the second user over the network 414; the transmission of content on virtual monitor A2 to the second user; and disconnecting from the second user. In this example embodiment, the virtual monitor B2 lists action items associated with the second virtual reality headset, including the connection with the first user over the network 404; the receipt of content from virtual monitor A2; and displaying the received content on virtual monitor B3. In some embodiments, there can be more than one collaborator. Thus, in some embodiments, many separate users collaborate at once, for example in a conference or a teacher with students and each participant can be located anywhere in the world.

In some embodiments, the first user uses the mouse cursor 408a to act on the content displayed on the shared virtual monitor A2. In other embodiments, the first user uses the control button 406a to maintain control over the content displayed by shared virtual monitor A2 such that the second user can only view the content displayed by the shared virtual monitor A2 on the virtual monitor B3 and cannot affect the content displayed by the shared virtual monitor A2. In an example embodiment, the first user uses the mouse cursor 408a to zoom on a certain region of the image displayed by shared virtual monitor A2 and the virtual monitor B3 displays the same zooming actions displayed on the shared virtual monitor A2. In other embodiments, the first user selects a zoom tool from a palette of tools (which also include linear measurements, density measurements, annotations—lines, circles, letters) and then can use the zoom tool—or whatever tool is selected, to pass control over the content displayed by both virtual monitors A2, B3 to the second user, such that the second user can use a mouse cursor or other tool to act over the content displayed by the virtual monitors A2, B3 while the first user can view the actions taken by the second user. In some of these embodiments, the same content is viewed in the two or more monitors viewed by the two or more users simultaneously (as far as human perception can determine). Although FIG. 4F through FIG. 4G depict two users of two virtual reality headsets connecting over a network, more than two users of more than two virtual reality headsets can connect over the network and communicate in a similar manner as the users discussed above.

As discussed above, in some embodiments, the second virtual reality headset has a different arrangement of virtual monitors 404b than the desired arrangement of virtual monitors 404a of the first virtual reality headset. In the example embodiment of FIG. 4F through FIG. 4G, the arrangement of virtual monitors 404b has a reduced quantity of monitors than the desired arrangement of virtual monitors 404a. As a result, some communication between the first user and second user over the network 414 is limited. For example, if the first user wanted to simultaneously share image content on the three displays A1, A2, A5, the second design space 402b cannot accommodate this share request, since only two of the virtual displays B1, B3 are designated to display image content. Thus, it would be advantageous to reconfigure the arrangement of virtual monitors 404b of the second virtual reality headset to match the arrangement of virtual monitors 404a of the first virtual reality headset upon connecting the virtual reality headsets of the first and second users over the network 414. FIG. 4H through FIG. 4I are block diagrams that illustrate an example of respective first and second matching design spaces 402a, 402b′ for first and second virtual reality headsets connected over a network 414, according to an embodiment. In some embodiments, the module 204 causes the controller 202 to transmit a signal with the desired arrangement of the virtual monitors 404a over the network 414 to a module (not shown) of a corresponding controller of a second system 200b. Upon receiving this signal, the module of the second system 200b stores the desired arrangement of the virtual monitors 404a in a memory of the controller and uses this arrangement to generate the design space 402b′ (step 303) corresponding to the design space 402a. As a result, if the first user wants to share the image content displayed on virtual monitors A1, A2, A5, the revised design space 402b′ can accommodate this request. What enables all this in some embodiments is that all information comes from a server 234, which is itself connected to the archive that stores all the data (images, reports, lab values, etc.). So all users can view data related to a particular patient or entity simultaneously.

### 2. Example Embodiments

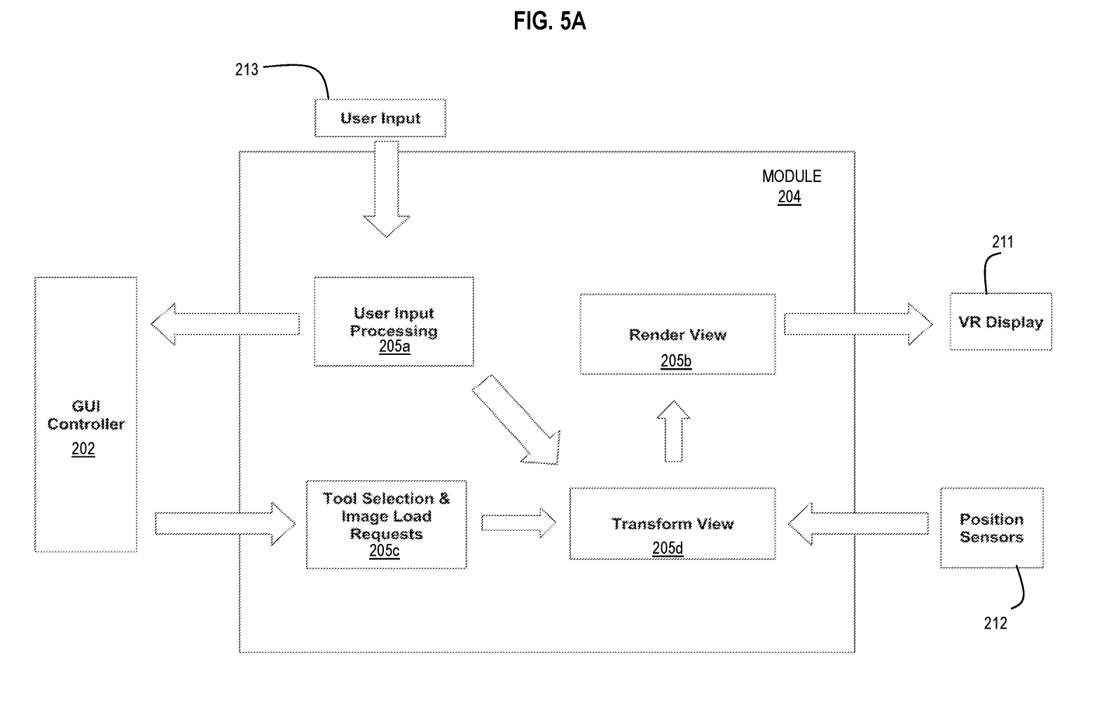

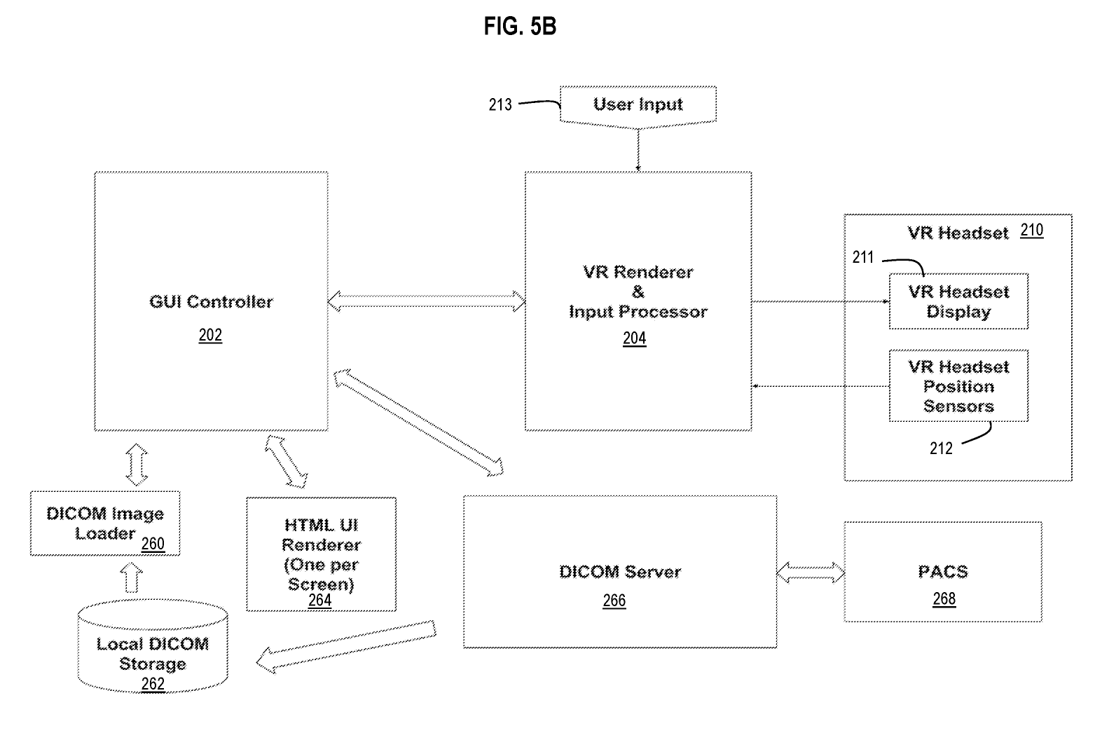

FIG. 5A is a block diagram that illustrates an example of data flow within the system of FIG. 2D, according to an embodiment. The input device 213 is used to provide user input of one or more parameters of the desired arrangement of virtual monitors 404 in the design space 402. In some embodiments, the module 204 includes a user input processing submodule 205a that receives (e.g. step 301) the user inputted parameters of the desired arrangement of the virtual monitors 404 in the design space 402. The user input processing submodule 205a processes the inputted parameters and generates a design space (step 303) which it then stores in memory of the controller 202. The user input processing submodule 205a also transmits a signal to the transform view submodule 205d with data of the design space 402.

Additionally, the head position sensor 212 provides input to the transform view submodule 205d (e.g. step 305) based on the one or more parameters related to a position or motion of the user 208 head. The transform view submodule 205d then determines a view space movement (e.g. step 307) based on the head movement. The transform view submodule 205d then moves the view space over the design space (step 309), based on the determined view space movement and the design space data received from the user input processing submodule 205a. The transform view submodule 205d then transmits a signal to the render view submodule 250b of the selective portion of the design space 402 corresponding to the moved view space 412. The render view submodule 205b then transmits a signal to the display 211 of the virtual reality headset 210, to present the selective portion of the design space 402 (step 311) corresponding to the moved view space 412.

Additionally, the controller 202 provides content data (e.g. image data) to be displayed on the virtual monitors 404 to a tool selection and image load request submodule 205c of the module 204. The submodule 205c transmits a signal to the transform view submodule 205d based on the received content data, and the transform view submodule 205d subsequently transmits a signal to the render view submodule 205b which in turn causes the display 211 of the virtual reality headset 210 to display the content data on the virtual monitors 404. Although the data flow diagram of FIG. 5A depicts that the module 204 includes various submodules 205a-205d, this is merely one example embodiment of the module 204.

FIG. 5B is a block diagram that illustrates an example of data flow within the system of FIG. 2D, according to an embodiment. The block diagram of FIG. 5B is similar to the block diagram of FIG. 5A, but further depicts various components that are used to store image data and communicatively coupled to the controller 202 including a Digital Imaging and Communications in Medicine (DICOM) server 266, a local DICOM storage 262 and a DICOM image loader 260. In some embodiments, image data is uploaded or downloaded directly from or to the DICOM server 266 to the controller 202. In other embodiments, image data is uploaded from or to the DICOM server 266 to the local DICOM storage 262 to the DICOM image loader 260 and subsequently to the controller 202. In other embodiments, the DICOM server is communicatively coupled to a picture archiving and communication system (PACS) 268. In another embodiment, the controller 202 downloads or uploads image data from a Hyper Text Markup Language (HTML) user interface (UI) renderer 264.

### 3. Computational Hardware

FIG. 6 is a block diagram that illustrates a computer system 600 upon which an embodiment of the invention may be implemented. Computer system 600 includes a communication mechanism such as a bus 610 for passing information between other internal and external components of the computer system 600. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 600, or a portion thereof, constitutes a means for performing one or more steps of one or more methods described herein.

A sequence of binary digits constitutes digital data that is used to represent a number or code for a character. A bus 610 includes many parallel conductors of information so that information is transferred quickly among devices coupled to the bus 610. One or more processors 602 for processing information are coupled with the bus 610. A processor 602 performs a set of operations on information. The set of operations include bringing information in from the bus 610 and placing information on the bus 610. The set of operations also typically include comparing two or more units of information, shifting positions of units of information, and combining two or more units of information, such as by addition or multiplication. A sequence of operations to be executed by the processor 602 constitutes computer instructions.

Computer system 600 also includes a memory 604 coupled to bus 610. The memory 604, such as a random access memory (RAM) or other dynamic storage device, stores information including computer instructions. Dynamic memory allows information stored therein to be changed by the computer system 600. RAM allows a unit of information stored at a location called a memory address to be stored and retrieved independently of information at neighboring addresses. The memory 604 is also used by the processor 602 to store temporary values during execution of computer instructions. The computer system 600 also includes a read only memory (ROM) 606 or other static storage device coupled to the bus 610 for storing static information, including instructions, that is not changed by the computer system 600. Also coupled to bus 610 is a non-volatile (persistent) storage device 608, such as a magnetic disk or optical disk, for storing information, including instructions, that persists even when the computer system 600 is turned off or otherwise loses power.

Information, including instructions, is provided to the bus 610 for use by the processor from an external input device 612, such as a keyboard containing alphanumeric keys operated by a human user, or a sensor. A sensor detects conditions in its vicinity and transforms those detections into signals compatible with the signals used to represent information in computer system 600. Other external devices coupled to bus 610, used primarily for interacting with humans, include a display device 614, such as a cathode ray tube (CRT) or a liquid crystal display (LCD), for presenting images, and a pointing device 616, such as a mouse or a trackball or cursor direction keys, for controlling a position of a small cursor image presented on the display 614 and issuing commands associated with graphical elements presented on the display 614.

In the illustrated embodiment, special purpose hardware, such as an application specific integrated circuit (IC) 620, is coupled to bus 610. The special purpose hardware is configured to perform operations not performed by processor 602 quickly enough for special purposes. Examples of application specific ICs include graphics accelerator cards for generating images for display 614, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 600 also includes one or more instances of a communications interface 670 coupled to bus 610. Communication interface 670 provides a two-way communication coupling to a variety of external devices that operate with their own processors, such as printers, scanners and external disks. In general the coupling is with a network link 678 that is connected to a local network 680 to which a variety of external devices with their own processors are connected. For example, communication interface 670 may be a parallel port or a serial port or a universal serial bus (USB) port on a personal computer. In some embodiments, communications interface 670 is an integrated services digital network (ISDN) card or a digital subscriber line (DSL) card or a telephone modem that provides an information communication connection to a corresponding type of telephone line. In some embodiments, a communication interface 670 is a cable modem that converts signals on bus 610 into signals for a communication connection over a coaxial cable or into optical signals for a communication connection over a fiber optic cable. As another example, communications interface 670 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 670 sends and receives electrical, acoustic or electromagnetic signals, including infrared and optical signals, that carry information streams, such as digital data.

The term computer-readable medium is used herein to refer to any medium that participates in providing information to processor 602, including instructions for execution. Such a medium may take many forms, including, but not limited to, non-volatile media, volatile media and transmission media. Non-volatile media include, for example, optical or magnetic disks, such as storage device 608. Volatile media include, for example, dynamic memory 604. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 602, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 602, except for carrier waves and other signals.

Logic encoded in one or more tangible media includes one or both of processor instructions on a computer-readable storage media and special purpose hardware, such as ASIC *620.

Network link 678 typically provides information communication through one or more networks to other devices that use or process the information. For example, network link 678 may provide a connection through local network 680 to a host computer 682 or to equipment 684 operated by an Internet Service Provider (ISP). ISP equipment 684 in turn provides data communication services through the public, world-wide packet-switching communication network of networks now commonly referred to as the Internet 690. A computer called a server 692 connected to the Internet provides a service in response to information received over the Internet. For example, server 692 provides information representing video data for presentation at display 614.

The invention is related to the use of computer system 600 for implementing the techniques described herein. According to one embodiment of the invention, those techniques are performed by computer system 600 in response to processor 602 executing one or more sequences of one or more instructions contained in memory 604. Such instructions, also called software and program code, may be read into memory 604 from another computer-readable medium such as storage device 608. Execution of the sequences of instructions contained in memory 604 causes processor 602 to perform the method steps described herein. In alternative embodiments, hardware, such as application specific integrated circuit 620, may be used in place of or in combination with software to implement the invention. Thus, embodiments of the invention are not limited to any specific combination of hardware and software.

The signals transmitted over network link 678 and other networks through communications interface 670, carry information to and from computer system 600. Computer system 600 can send and receive information, including program code, through the networks 680, 690 among others, through network link 678 and communications interface 670. In an example using the Internet 690, a server 692 transmits program code for a particular application, requested by a message sent from computer 600, through Internet 690, ISP equipment 684, local network 680 and communications interface 670. The received code may be executed by processor 602 as it is received, or may be stored in storage device 608 or other non-volatile storage for later execution, or both. In this manner, computer system 600 may obtain application program code in the form of a signal on a carrier wave.

Various forms of computer readable media may be involved in carrying one or more sequence of instructions or data or both to processor 602 for execution. For example, instructions and data may initially be carried on a magnetic disk of a remote computer such as host 682. The remote computer loads the instructions and data into its dynamic memory and sends the instructions and data over a telephone line using a modem. A modem local to the computer system 600 receives the instructions and data on a telephone line and uses an infra-red transmitter to convert the instructions and data to a signal on an infra-red a carrier wave serving as the network link 678. An infrared detector serving as communications interface 670 receives the instructions and data carried in the infrared signal and places information representing the instructions and data onto bus 610. Bus 610 carries the information to memory 604 from which processor 602 retrieves and executes the instructions using some of the data sent with the instructions. The instructions and data received in memory 604 may optionally be stored on storage device 608, either before or after execution by the processor 602.

FIG. 7 illustrates a chip set 700 upon which an embodiment of the invention may be implemented. Chip set 700 is programmed to perform one or more steps of a method described herein and includes, for instance, the processor and memory components described with respect to FIG. *6 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 700, or a portion thereof, constitutes a means for performing one or more steps of a method described herein.

In one embodiment, the chip set 700 includes a communication mechanism such as a bus 701 for passing information among the components of the chip set 700. A processor 703 has connectivity to the bus 701 to execute instructions and process information stored in, for example, a memory 705. The processor 703 may include one or more processing cores with each core configured to perform independently. A multi-core processor enables multiprocessing within a single physical package. Examples of a multi-core processor include two, four, eight, or greater numbers of processing cores. Alternatively or in addition, the processor 703 may include one or more microprocessors configured in tandem via the bus 701 to enable independent execution of instructions, pipelining, and multithreading. The processor 703 may also be accompanied with one or more specialized components to perform certain processing functions and tasks such as one or more digital signal processors (DSP) 707, or one or more application-specific integrated circuits (ASIC) 709. A DSP 707 typically is configured to process real-world signals (e.g., sound) in real time independently of the processor 703. Similarly, an ASIC 709 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 703 and accompanying components have connectivity to the memory 705 via the bus 701. The memory 705 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 705 also stores the data associated with or generated by the execution of one or more steps of the methods described herein.

### 4. Modifications and Alterations

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article. As used herein, unless otherwise clear from the context, a value is “about” another value if it is within a factor of two (twice or half) of the other value. While example ranges are given, unless otherwise clear from the context, any contained ranges are also intended in various embodiments. Thus, a range from 0 to 10 includes the range 1 to 4 in some embodiments.

1. A method comprising:
receiving, on a processor, a design space describing a desired arrangement of virtual monitors;
receiving, on the processor, data associated with head movement of a user wearing a virtual reality headset;
determining, on the processor, a movement of a view space over the design space wherein the view space encompasses only a portion of the design space, wherein the movement of the view space is based on the head movement such that a ratio of the view space movement to the head movement is in a range comprising values other than unity;
moving the view space over the design space based on the determined movement of the view space; and
presenting on the virtual reality headset the portion of the design space within the view space.

2. The method of claim 1, further comprising:
receiving, on the processor, an input of the desired arrangement of virtual monitors; and
generating the design space including the desired arrangement of the virtual monitors.

3. The method of claim 1, wherein the range is preset or programmable.

4. The method of claim 2, wherein the range is based on an input other than the desired arrangement of the virtual monitors.

5. The method of claim 1, wherein the range values comprise 50-150%.

6. The method of claim 2, further comprising inputting the desired arrangement of virtual monitors with an input device, said desired arrangement including at least one of a number of virtual monitors in the desired arrangement, a size of each virtual monitor, and a position of each virtual monitor in the desired arrangement.

7. The method of claim 6, further comprising inputting a modification to the desired arrangement of virtual monitors with the input device, said modification to the desired arrangement including at least one of a modification to the number of virtual monitors, a modification to the size of at least one virtual monitor, and a modification to the position of at least one virtual monitor.

8. The method of claim 1, further comprising selecting a specific virtual monitor in the view space with an input device and affecting content on only the specific virtual monitor with the input device.

9. The method of claim 1, further comprising:
connecting the virtual reality head set to a second virtual reality head set configured to be worn by a second user over a network, said second virtual reality head set including a second arrangement of virtual monitors different than the desired arrangement of virtual monitors;
selecting at least one virtual monitor in the view space with an input device; and
enabling the second user to view content on the at least one selected virtual monitor over the network.

10. The method of claim 9, further comprising reconfiguring the second arrangement of virtual monitors to match the desired arrangement of virtual monitors upon connecting the virtual reality head set to the second virtual reality head set over the network.

11. The method of claim 9, further comprising enabling the second user to affect content on the at least one selected virtual monitor over the network.

12. The method of claim 9, further comprising preventing the second user from viewing content on the virtual monitors other than the selected virtual monitor over the network.

13. An apparatus comprising:
a virtual reality headset configured to be worn on a user's head;
at least one processor; and
at least one memory including one or more sequences of instructions;
the at least one memory and the one or more sequences of instructions configured to, with the at least one processor, cause the apparatus to perform at least the following, receive a design space describing a desired arrangement of virtual monitors;
receive data associated with head movement of the user wearing the virtual reality headset;
determine a movement of a view space over the design space wherein the view space encompasses only a portion of the design space, wherein the movement of the view space is based on the head movement such that a ratio of the view space movement to the head movement is in a range comprising values other than unity;
move the view space over the design space based on the determined movement of the view space; and
present on the virtual reality headset the portion of the design space within the view space.

14. The apparatus of claim 13, wherein the at least one memory and sequences of instructions, with the at least one processor is further configured to cause the apparatus to:
receive an input of the desired arrangement of the virtual monitors; and
generate the design space including the desired arrangement of virtual monitors.

15. The apparatus of claim 13, wherein the range is preset or programmable.

16. The apparatus of claim 14, wherein the range is based on an input other than the desired arrangement of the virtual monitors.

17. The apparatus of claim 13, wherein the range values comprise 50-150%.

18. The apparatus of claim 14, further comprising an input device configured for the user to provide the input of the desired arrangement of virtual monitors, wherein the desired arrangement includes at least one of a number of virtual monitors in the desired arrangement, a size of each virtual monitor, and a position of each virtual monitor in the desired arrangement.

19. The apparatus of claim 18, wherein the input device is further configured for the user to provide a modification to the input of the desired arrangement of virtual monitors, wherein the modification to the desired arrangement includes at least one of a modification to the number of virtual monitors, a modification to the size of at least one virtual monitor, and a modification to the position of at least one virtual monitor.

20. The apparatus of claim 13, further comprising an input device configured for the user to select a specific virtual monitor in the view space, and wherein the at least one memory and sequences of instructions, with the at least one processor is further configured to cause the input device to only affect content on the specific virtual monitor.

21. The apparatus of claim 13, further comprising:
a second virtual reality headset configured to be worn on a second user's head, said second virtual reality headset connected to the virtual reality headset over a network and including a second arrangement of virtual monitors different than the desired arrangement of virtual monitors; and
an input device configured to select at least one virtual monitor in the view space;
and wherein the at least one memory and sequences of instructions, with the at least one processor is further configured to enable the second user to view content on the at least one selected virtual monitor over the network.
